(19) United States
(12) Patent Application Publication
(10) Pub. No.: US 2020/0113518 A1
(43) Pub. Date: Apr. 16, 2020

(54) SYSTEM FOR FACILITATING MONITORING OF FITNESS DEVICES

(71) Applicant: Joshua Mollohan, Castle Rock, CO (US)

(72) Inventor: Joshua Mollohan, Castle Rock, CO (US)

(21) Appl. No.: 16/158,627

(22) Filed: Oct. 12, 2018

(51) Int. Cl. A61B 5/00; A63B 24/00; G16H 20/60

(52) U.S. Cl. CPC A61B 5/681; A61B 5/222; G16H 20/60; A63B 24/0062

(57) ABSTRACT

A system for facilitating monitoring of fitness devices is disclosed. The system may include a plurality of sensors configured to be attached to a plurality of fitness devices. Further, the system may include a plurality of transceivers communicatively coupled with the plurality of sensors. Further, the system may include a plurality of local processing devices communicatively coupled to each of the plurality of sensors and the plurality of transceivers. Further, the system may include a server computer. The server computer may include a server communication device configured for communicating with the at least one transceiver. Further, the server computer may include a server processing device communicatively coupled with the server communication device. Further, the server processing device may be configured for analyzing the sensor data and generating at least one insight. Further, the server computer may include a server storage device communicatively coupled to the server processing device.

### FIELD OF THE INVENTION

The present invention relates generally to fitness devices. More specifically, the present disclosure describes an integrated system for allowing health clubs and patrons to easily track workouts and diets.

### BACKGROUND OF THE INVENTION

Across the world, especially in the US, obesity is a growing epidemic. Over the past few decades, obesity has skyrocketed. It's costing billions in healthcare, as it is five times more expensive to insure someone who is obese than someone who smokes. Even companies are seeing an increase in lost productivity do to this epidemic.

The current methods available for living a healthier lifestyle and achieving desired results are ineffective. Many companies are looking for better wellness programs through health clubs to help assist their employees in living a healthier lifestyle. For many, being overweight or obese is a large concern, as evidenced by large spending on health related goods. Diet pills, weight loss plans, and gym memberships appeal to people who want to improve their health. These people spend a significant money on such goals, though often these fad diets and trends do not create lasting health benefits.

While some are able to achieve their health goals, many others face challenges. A common occurrence is the “yo-yo dieter”, someone who will shed weight only to later put it back on. This cycle continues on for many people who may become discouraged and give up after repeated failures. However, for holistic wellness, the weight loss and improved health needs to be maintained. In order to correct this trend, people need to think about lifestyle changes, not temporary diets; the bottom line is, if a person returns to their unhealthy lifestyle after achieving their goals, weight will pile back on.

To better understand the nature of wellness, simply look at what a challenge it is to keep track of calories in and out. Many people struggle with “cheating” by eating extra snacks and underestimating consumed calories. A simple way to combat this is for people to keep logs of exercises and food consumed. By providing a written record of caloric intake and expenditure, a person is better able to adhere to their nutritional guidelines for achieving a healthier body. Helping with this process, more and more restaurants choose to provide calorie estimates for menu options. Recent federal laws have changed such that restaurants with 20 or more locations must provide nutritional information. This is helpful for those who track calories; however, there are still those who have difficulty maintaining a record of food intake. While there are programs which work to help people adhere to nutritional requirements, such as by a points system, there remains a need for a system that allows persons to effortlessly record exercise and nutritional data to better achieve their health goals. One of the most effective ways to achieve these desired results is through accountability and multi point verification processes.

Therefore, there is a need for improved systems for facilitating monitoring of fitness devices and nutritional intake, that may overcome one or more of the above-mentioned problems and/or limitations.

### SUMMARY

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

According to some embodiments, a system for facilitating monitoring of fitness devices is disclosed. The system may include a plurality of sensors configured to be attached to a plurality of fitness devices. Further, the plurality of sensors may be configured for generating sensor data representative of at least one state of the plurality of fitness devices. Further, the system may include a plurality of transceivers communicatively coupled with the plurality of sensors. Further, the plurality of transceivers may be further configured for communicating with each of a portable electronic device and a server computer. Further, the system may include a plurality of local processing devices communicatively coupled to each of the plurality of sensors and the plurality of transceivers. Further, the plurality of local processing devices may be further configured for processing the sensor data. Further, the system may include a server computer. The server computer may include a server communication device configured for communicating with the at least one transceiver. Further, the communicating may include receiving the sensor data. Further, the server computer may include a server processing device communicatively coupled with the server communication device. Further, the server processing device may be configured for analyzing the sensor data. Further, the server processing device may be configured for generating at least one insight corresponding to at least one fitness device of the plurality of fitness devices based on the analyzing of the sensor data. Further, the server computer may include a server storage device communicatively coupled to the server processing device. Further, the server storage device may be configured for storing the sensor data and the at least one insight in association with a plurality of fitness device identifiers associated with the plurality of fitness devices.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 is an illustration of an online platform consistent with various embodiments of the present disclosure.

FIG. 2 is a block diagram of the system for facilitating monitoring of fitness devices, in accordance with various embodiments of the present disclosure.

FIG. 3 is a block diagram of the system for facilitating monitoring of fitness devices, in accordance with various embodiments of the present disclosure.

FIG. 4 is a block diagram of the system for facilitating monitoring of fitness devices, in accordance with various embodiments of the present disclosure.

FIG. 5 is a block diagram of the system for facilitating monitoring of fitness devices, in accordance with various embodiments of the present disclosure.

FIG. 6 is a block diagram of the system for facilitating monitoring of fitness devices, in accordance with various embodiments of the present disclosure.

FIG. 7 is a block diagram of the system for facilitating monitoring of fitness devices, in accordance with various embodiments of the present disclosure.

FIG. 8 is a block diagram of a system for integrating technology for health and fitness, linking together health clubs, patrons, and restaurants for the benefit of all parties involved, in accordance with various embodiments of the present disclosure.

FIG. 9 is a flowchart of a method for recording workout in gym in accordance with an exemplary embodiment.

FIG. 10 is a flowchart of a method for using gym equipment in accordance with an exemplary embodiment.

FIG. 11 is a flowchart of a method for recording workout outside a gym in accordance with an exemplary embodiment.

FIG. 12 is a flowchart of a method for providing food choices to a user in accordance with an exemplary embodiment.

FIG. 13 is a flowchart of a method for customizing workouts for a user in accordance with an exemplary embodiment.

FIG. 14 illustrates a treadmill that may be placed in a health club in accordance with an exemplary embodiment.

FIG. 15 shows a screen, a fob and a sensor for gym equipment in accordance with an exemplary embodiment.

FIG. 16 illustrates a weight plate with a sensor in accordance with an exemplary embodiment.

FIG. 17 illustrates a side view of a plates loaded machine with a fob in accordance with an exemplary embodiment.

FIG. 18 illustrates a dumbbell with a fob in accordance with an exemplary embodiment.

FIG. 19 illustrates a floor map of a gym in accordance with an exemplary embodiment.

FIG. 20 is a block diagram of a computing device for implementing the methods disclosed herein, in accordance with some embodiments.

### DETAIL DESCRIPTIONS OF THE INVENTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of monitoring fitness devices, embodiments of the present disclosure are not limited to use only in this context.

Overview

According to some embodiments, a system is provided that may be retro-fitted to existing equipment in health club/gyms. Each club may have its own system and network which may communicate with other clubs under the same ownership or chain.

Further, the disclosure is related to an automated tracking system for health clubs and both home and commercial gyms. A software application installed on the phone and or smart watch may be used to store the user personal information, weight, age sex and over all goals. The patron is logged in automatically once they enter in the facility (such as a gym). The application may ask if the user would like to select a workout based on their goals. If they select yes, then the application with use geolocation may direct them to the workout equipment. Every piece of equipment (dumbbells, free weights, machine weights, cardio equipment, apparatuses, kettlebells or any other equipment found in clubs) may be outfitted with an informational fob. Once the user is within a couple feet of gym equipment, the application may sync with the equipment. They may start their workout once they are synced-up. The fob may be equipped with accelerometer, motion detection, Bluetooth and Wi-Fi. The user may also select a video tutorial to show them how to use the equipment. As the user works out their repetitions, sets, rest periods, the caloric expenditure will be automatically tracked and stored with in their profile. Once that exercise is completed they will be directed towards the next exercise. At the end of their workout the user(s) will be able to see their progress. The application may use this information to provide suggestions on future workouts based on their goals. However, if a patron chooses no when asked if they want to do an already created workout, they will go into manual mode. All functionality may be the same and the application may automatically track the workouts them same.

Further, the gym owners may be able to track what machines are being utilized more than others, what machines require more maintenance than others etc. The application may also provide a digital map of traffic within the club. This may allow gym owners to decide where they should place advertisements with in the club based on the traffic of members with in the club. The application may also be able verify workouts done by the user for personal trainers to review and also wellness programs that other businesses participate in with the health club/gym. The application also has the ability to interact with existing nutritional application(s).

Further, the disclosed system provides tools to gym owners to create a better user experience for its patrons. For example, the system may help patrons accomplish their health goals by providing them suggestions regarding the workouts and diets to follow. Further, the system may automatically reduce wait times of patrons at various machines. This may result in better results for patrons, which in return will lead to better retention.

Further, each machine in the club may be equipped with a screen that has a motion sensor, camera, fingerprint reader, an active ultra-bandwidth RFID, an accelerometer, a gyroscope, Wi-Fi capability, and Bluetooth capability.

Accordingly, when a patron comes to the club they may select the workout they would like to do via the application. The application may show if machines are available (motion sensor and cameras near the machines may report to the network if the machines are being used) for the workout and via gym mapping (via active ultra-bandwidth RFID, which is accurate to within a few centimeters) where each piece of equipment is located. The users may see a video (via the screen or the application) showing how to use each machine or perform an exercise. It will automatically log repetitions, sets and rest periods (via accelerometer and gyroscope). It will give feedback and suggestions for future workouts based on progress and goals.

If the desired machine is being used the application, it may provide alternative exercises/machines that are available. If the patron really wants to use the piece of equipment that is unavailable, they may request for the same. If current weight machine user is on the machine longer than 10-15 minutes (the gym owner may select this timeframe) they may see a message/icon on the screen indicating that someone is waiting to use the machine.

Further, if the patron does not have or forgets their phone they may still log their workouts. They may quickly and easily login their workouts to their account via fingerprint scanner or face recognition (fingerprint sensor and camera). Their workout will be uploaded to their account via a gym network.

The disclosed system may have multiple point verification (membership check-in, fingerprint and or face recognition) for participation in wellness programs and for personal trainers to review. This ensures workouts have actually been completed by that person and the patron didn't simply have a friend record the workouts by using patrons phone/smartwatch to log workouts to receive credit for enrolled programs and contest.

Further, the equipment may have automated maintenance request abilities. For example, after every thousand repetitions on weight machines, the owner/maintenance may be notified that the machine needs to be serviced. Further, the cardio equipment may make an automatic service request for every 150 miles of use. A patron may also report a machine not properly working (via the screen). Accordingly, the owner/maintenance may more effectively and efficiently maintain equipment based on the usage and popularity. This may also help determine the depreciation of equipment. Further, the owner/maintenance may also log what maintenance (via screen) that has been done or needs to be done and order parts for the machines. This may assist in keeping machines running longer and expedite repairs so machines are not out of service and are fixed faster.

According to some embodiments, a user may use the application to create an account. The user may provide information including one or more of age, sex, weight, height, and goals (weight loss, weight gain, etc.) while creating the account. Further, the application may take that information and process it through the Harris-Benedict formula (refer equations 1 and 2 below) to calculate your BMR (basal metabolic rate).

Women: BMR=655+(4.35×weight in pounds)+(4.7×height in inches)−(4.7×age in years) (1)

Men: BMR=66+(6.23×weight in pounds)+(12.7×height in inches)−(6.8×age in years) (2)

Thereafter, the application may multiple the BMR with an appropriate activity factor, as described using equations 3-7 below:

Sedentary (little or no exercise): BMR×1.2 (3)

Lightly active (light exercise/sports 1-3 days/week): BMR×1.375 (4)

Moderately active (moderate exercise/sports 3-5 days/week): BMR×1.55 (5)

Very active (hard exercise/sports 6-7 days a week): BMR×1.725 (6)

Extra active (very hard exercise/sports & physical job or 2× training): BMR×1.9 (7)

The final number obtained using equations 3-7 above is the approximate number of calories required each day to maintain the weight of a user.

Further, based on the goals, the application may adjust the calories needed. The application may also break down those needs into macronutrients protein, fats, and carbohydrates. Accordingly, the application may take the calories/macronutrients (for example, 2000 calories) that are required for a goal and break those down into 3 meals and 2 snacks (3—500-calories-meals and 2—250-calorie-snack) to help their metabolism and energy levels.

Further, the application may then set reminders and notifications for when to eat based on the daily habits (such as what time users wakes up or workouts). Further, the users may also have the ability to search local menus to meet their nutritional requirements

Further, the application may obtain restaurant menus. Accordingly, the application may obtain a geo-location of restaurants within a specified radius. Then, the application may take those restaurants and cross-reference them with the nutritional database. Next, the application may apply a filter based on caloric/nutritional requirements and narrow what is available based on those requirements. Then, the application may inform how many options are available at each location and what they may include. Alternatively, the user may use the application to sort and search menus by calories and or macro-nutrients. For example, the user may search menus for under 50 calorie protein rich meals.

Then, once the user selects the restaurant they want to go to, the application may provide directions to the corresponding location. Further, once the user reaches the location, the application may obtain information such as what they are eating. This information may be automatically loaded to a nutritional journal.

Similarly, grocery stores may be searched. The nutritional database may be used to find nutritional information by scanning the barcode on an item or by word search of the item and automatically load the selection into the nutritional journal.

According to some embodiments, the system may provide an integrated gym wherein the nutritional function works in conjunction with the workouts function. The application makes adjustments to a workout program based on the goals and how a user is doing on their nutritional program. For example, if the user is over 100 calories for the day, the application may make a suggestion to add 20 minutes of cardio to make up for the excess in calories. Further, the user may also load in exercises done outside of the gym. There is an exercise database to select what they did and automatically load that information into their exercise journal. Further, the system may use machine learning and artificial intelligence technologies to be able suggest workouts and changes to the diet as well.

According to some embodiments, the present disclosure provides an integrated system that allows a health club patron to have their exercise data automatically updated to cloud storage by means of an interactive health watch, smartphone application, and informational fob. Further, the present disclosure provides means for a person to easily scan nutritional information from barcodes, with their daily consumption being automatically loaded to cloud storage.

The system will rely heavily on machine learning and artificial intelligence. That will be necessary for it to be able suggest workouts and changes to the diet as well.

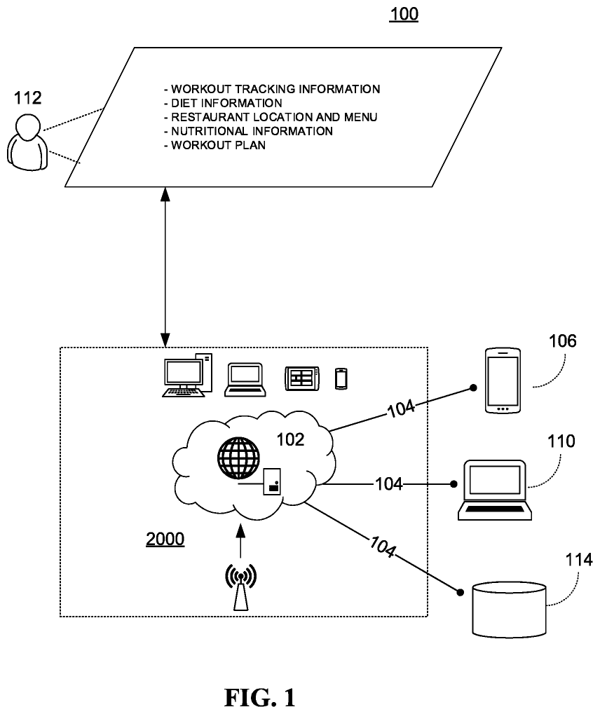

FIG. 1 is an illustration of an online platform 100 consistent with various embodiments of the present disclosure. By way of non-limiting example, the online platform 100 for facilitating monitoring of fitness devices may be hosted on a centralized server 102, such as, for example, a cloud computing service. The centralized server 102 may communicate with other network entities, such as, for example, a mobile device 106 (such as a smartphone, a laptop, a tablet computer etc.), other electronic devices 110 (such as desktop computers, server computers etc.), databases 114 (such as nutritional database, restaurant's menu database, exercise database etc.), over a communication network 104, such as, but not limited to, the Internet. Further, users of the online platform 100 may include relevant parties such as, but not limited to, end users, and administrators. Accordingly, in some instances, electronic devices operated by the one or more relevant parties may be in communication with the platform 100.

A user 112, such as the one or more relevant parties, may access online platform 100 through a web-based software application or browser. The web-based software application may be embodied as, for example, but not be limited to, a website, a web application, a desktop application, and a mobile application compatible with a computing device 2000.

According to some embodiments, the online platform 100 may be configured to operate in conjunction with a system 200 for facilitating monitoring of fitness devices. The system 200 is explained in detail below.

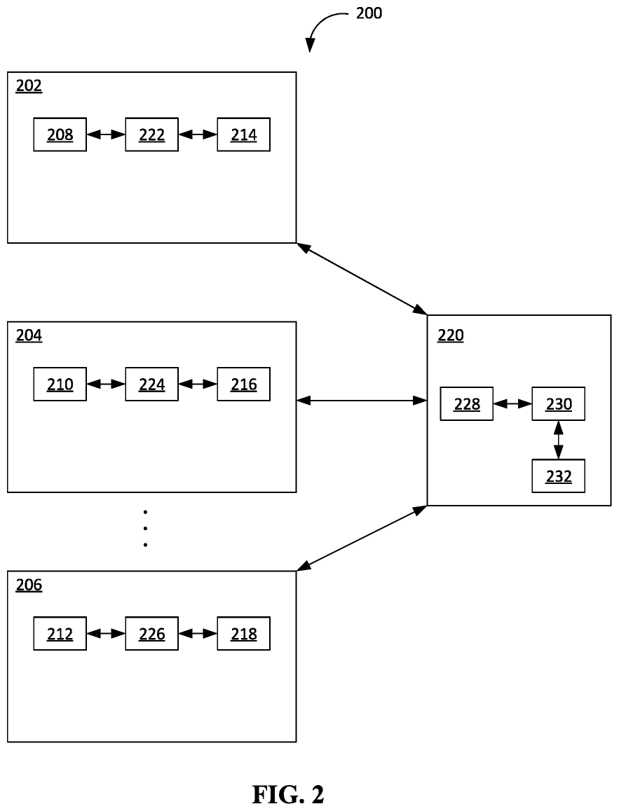

FIG. 2 is a block diagram of the system 200 for facilitating the monitoring of fitness devices, in accordance with various embodiments of the present disclosure. The system 200 may include a plurality of sensors 208-212 configured to be attached to a plurality of fitness devices 202-206. Further, the plurality of sensors 208-212 may be configured for generating sensor data representative of at least one state of the plurality of fitness devices 202-206.

Further, the system 200 may include a plurality of transceivers 214-218 communicatively coupled with the plurality of sensors 208-212. Further, the plurality of transceivers 214-218 may be further configured for communicating with each of a portable electronic device (not shown) and a server computer 220.

Further, the system 200 may include a plurality of local processing devices 222-226 communicatively coupled to each of the plurality of sensors 208-212 and the plurality of transceivers 214-218. Further, the plurality of local processing devices 222-226 may be further configured for processing the sensor data.

Further, the system 200 may include the server computer 220. The server computer 220 may include a server communication device 228 configured for communicating with the at least one transceiver. Further, the communicating may include receiving the sensor data. Further, the server computer 220 may include a server processing device 230 communicatively coupled with the server communication device 228. Further, the server processing device 230 may be configured for analyzing the sensor data. Further, the server processing device 230 may be configured for generating at least one insight corresponding to at least one fitness device of the plurality of fitness devices 202-206 based on the analyzing of the sensor data. Further, the server computer 220 may include a server storage device 232 communicatively coupled to the server processing device 230. Further, the server storage device 232 may be configured for storing the sensor data and the at least one insight in association with a plurality of fitness device identifiers associated with the plurality of fitness devices 202-206.

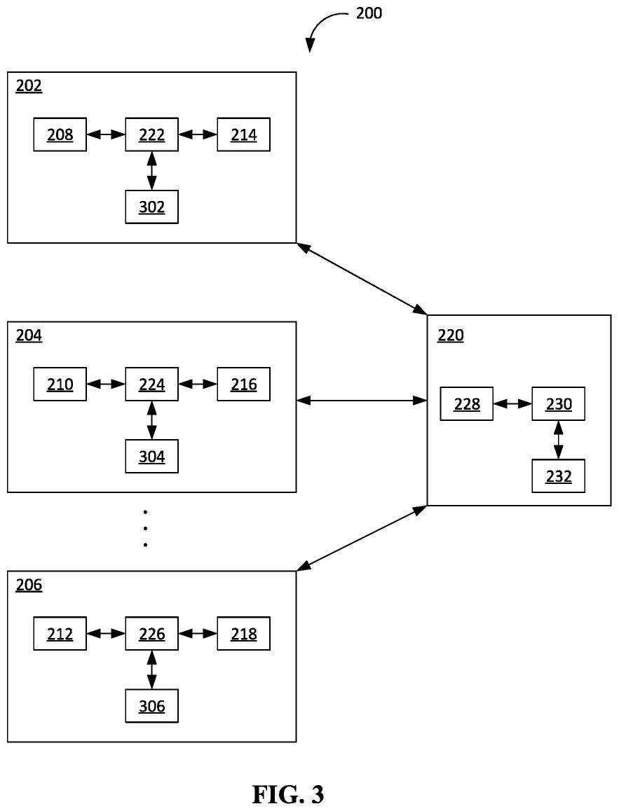

FIG. 3 is a block diagram of the system 200 for facilitating the monitoring of fitness devices, in accordance with further embodiments. The system 200 may further include a plurality of display devices 302-306 communicatively coupled to the plurality of processing devices. Further, the plurality of display devices 302-306 may be configured for displaying fitness data associated with the portable electronic device. Further, the plurality of transceivers 214-218 may be configured for receiving the fitness data from the portable electronic device.

In some embodiments, a local processing device of the plurality of local processing devices 222-226 may be configured for determining a usage time of a fitness device corresponding to the local processing device and generating a user waiting message based on the usage time exceeding a predetermined time period and receipt of a usage request. Further, a transceiver of the plurality of transceivers 214-218 corresponding to the fitness device may be further configured for receiving the usage request from one or more of the server computer 220 and at least one other transceiver of the plurality of transceivers 214-218. Further, a display device of the plurality of display devices 302-306 corresponding to the fitness device may be further configured for displaying the user waiting message.

In some embodiments, the at least one state of the plurality of fitness devices 202-206 may include one or more of an operational state and a usage state.

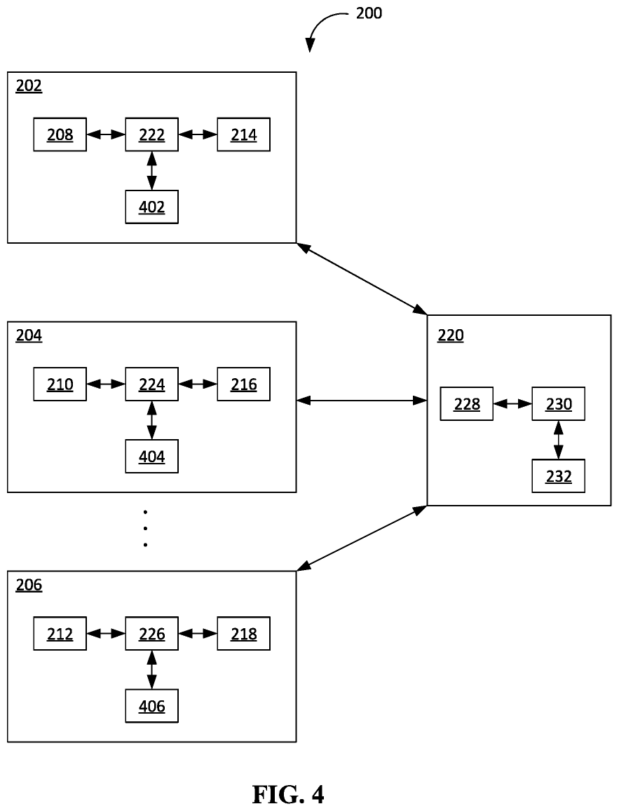

FIG. 4 is a block diagram of the system 200 for the facilitating monitoring of fitness devices, in accordance with further embodiments. The system 200 may further include a plurality of physiological sensors 402-406 corresponding to the plurality of fitness devices 202-206 communicatively coupled to the plurality of processing devices. Further, the plurality of physiological sensors 402-406 may be configured for generating physiological data corresponding to at least one user. Further, a physiological sensor corresponding to a fitness device may be configured for generating physiological data of a user of the fitness device during usage of the fitness device. Further, the plurality of transceivers 214-218 may be configured for transmitting the physiological data to one or more of the portable electronic device and the server computer 220.

In some embodiments, the plurality of transceivers 214-218 may be configured for transmitting the physiological data to the server computer 220. Further, the server processing device 230 may be further configured for analyzing the physiological data. Further, generating the at least one insight may be further based on the analyzing of the physiological data. Further, the server storage device 232 may be further configured for storing the physiological data in association with a user identifier associated with the user and a fitness device identifier associated with the fitness device.

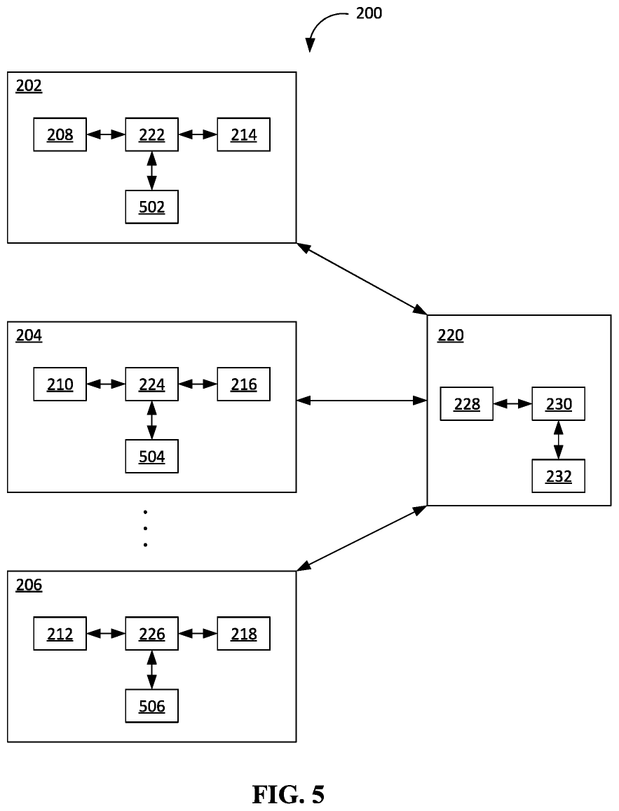

FIG. 5 is a block diagram of the system 200 for facilitating the monitoring of fitness devices, in accordance with further embodiments. In some embodiments, the system 200 may further include a plurality of biometric sensors 502-506 corresponding to the plurality of fitness devices 202-206 communicatively coupled to the plurality of local processing devices 222-226. Further, the plurality of biometric sensors 502-506 may be configured for generating biometric data associated with at least one user of the plurality of fitness devices 202-206. Further, the plurality of transceivers 214-218 may be configured for transmitting the biometric data to the server computer 220.

In some embodiments, the plurality of transceivers 214-218 may be further configured for receiving user identity data from the portable electronic device. Further, the plurality of transceivers 214-218 may be further configured for transmitting the user identity data to the server computer 220. Further, the plurality of transceivers 214-218 may be further configured for transmitting the biometric data associated with a user to the server computer 220. Further, the plurality of transceivers 214-218 may be further configured for receiving an authentication result from the server computer 220. Further, the server processing device 230 may be configured for authenticating the user based on the biometric data and the user identity. Further, the generating of the at least one insight may be further based on the authenticating.

In some embodiments, the plurality of transceivers 214-218 may be configured for transmitting a check-in indicator to the server computer 220 based on the communicating between the portable electronic device and at least one transceiver of the plurality of transceivers 214-218. Further, the check-in indicator represents entry of a user associated with the portable electronic device into a facility housing the plurality of fitness devices 202-206.

In some embodiments, the portable device may include a wearable computing device (not shown). Further, the wearable computing device may include at least one activity sensor configured for generating at least one physical activity data corresponding to a user wearing the wearable computing device. Further, the plurality of transceivers 214-218 may be further configured for receiving the at least one physical activity data and transmitting the at least one physical activity data to the server computer 220. Further, the generating of the at least one insight may be further based on the at least one physical activity data.

In some embodiments, the wearable computing device further configured for storing the at least one physical data corresponding to a first time period. Further, the sensor data corresponds to a second time period. Further, the first time period and the second time period may be non-identical. Further, the generating of the at least one insight may be further based on each of the first time period and the second time period.

In some embodiments, the plurality of transceivers 214-218 may be further configured for receiving a workout recommendation from the server computer 220. Further, the server processing device 230 may be further configured for generating the workout recommendation based on the at least one insight; transmitting the workout recommendation to the portable electronic device. Further, the portable electronic device may be further configured for presenting the workout recommendation.

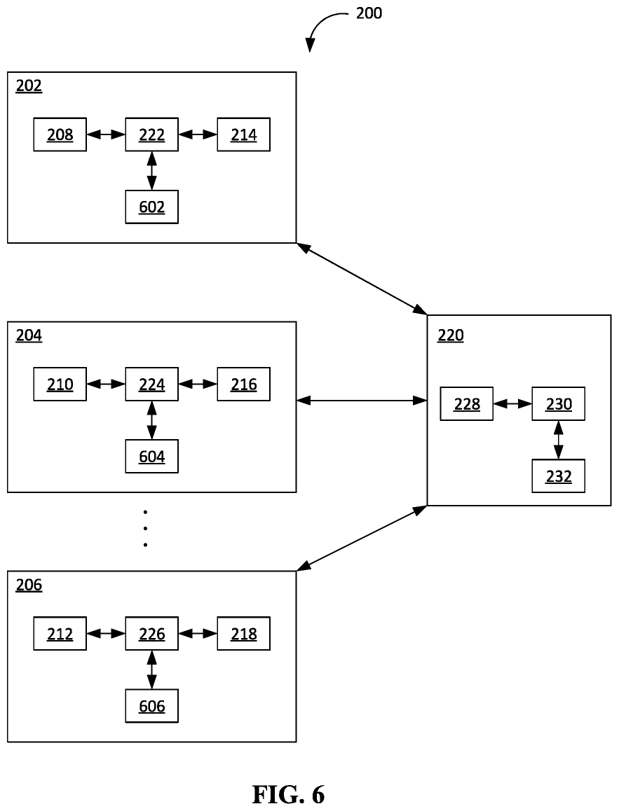

FIG. 6 is a block diagram of the system 200 for facilitating the monitoring of fitness devices, in accordance with further embodiments. The system 200 may further include a plurality of local storage devices 602-606 corresponding to the plurality of fitness devices 202-206. Further, the plurality of local storage devices 602-606 may be communicatively coupled to the plurality of local processing devices 222-226. Further, each local storage device of the plurality of local storage devices 602-606 may be configured for storing one or more of a location of a corresponding fitness device within a facility housing the plurality of fitness devices 202-206 and a fitness device identifier of the corresponding fitness device.

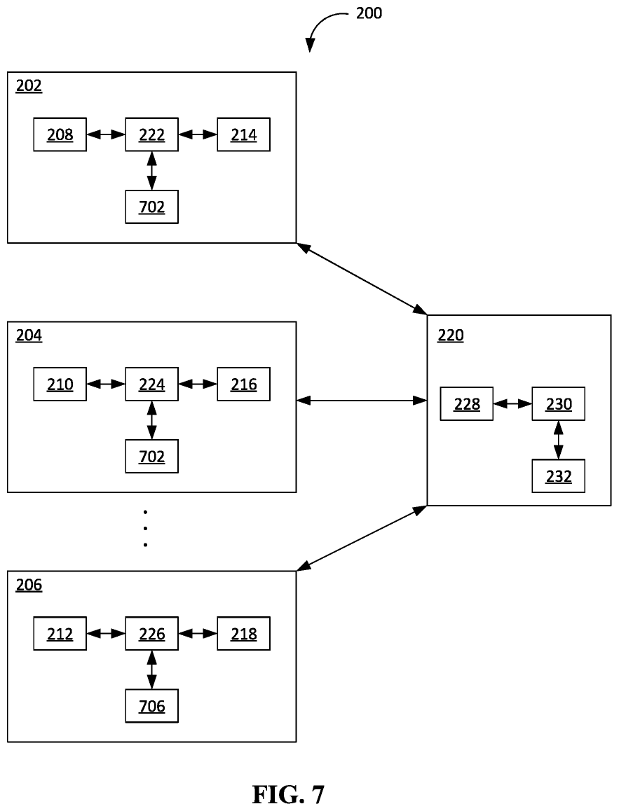

FIG. 7 is a block diagram of the system 200 for facilitating the monitoring of fitness devices, in accordance with further embodiments. The system 200 may further include a plurality of location sensors 702-706 corresponding to the plurality of fitness devices 202-206. Further, the plurality of location sensors 702-706 may be communicatively coupled to the plurality of local processing devices 222-226. Further, each location sensor of the plurality of location sensors 702-706 may be configured for generating the location of a corresponding fitness device.

In some embodiments, the plurality of local storage devices 602-606 may be further configured for storing a plurality of fitness device usage data. Further, the plurality of transceivers 214-218 may be further configured for transmitting the plurality of fitness device usage data to the portable device. Further, a fitness device usage data corresponding to a fitness device may include information pertaining to usage of the fitness device.

In some embodiments, the plurality of transceivers 214-218 may be configured for receiving a fitness goal from the portable electronic device. Further, the plurality of local processing devices 222-226 may be configured for generating a fitness device guidance data based on the fitness goal. Further, the plurality of transceivers 214-218 may be further configured for transmitting the fitness device guidance data to the portable electronic device. Further, a fitness device guidance data corresponding to a fitness device may include a location of the fitness device within the facility housing the plurality of fitness devices 202-206.

In some embodiments, the at least one insight may include at least one facility recommendation.

In some embodiments, the at least one facility recommendation may include a maintenance recommendation corresponding to at least one fitness device of the plurality of fitness devices 202-206.

In some embodiments, the server processing device 230 may be further configured for generating a maintenance request based on the maintenance recommendation. Further, the server communication device 228 may be further configured for transmitting the maintenance request to a service technician device.

In some embodiments, the system 200 may further include a plurality of facility display devices installed within the facility housing the plurality of fitness devices 202-206. Further, the plurality of facility display devices may be configured for receiving display content from the server computer 220. Further, the server processing device 230 may be further configured for generating the display content based on the at least one insight. Further, the at least one insight may include a traffic of a plurality of users within a facility housing the plurality of fitness devices 202-206.

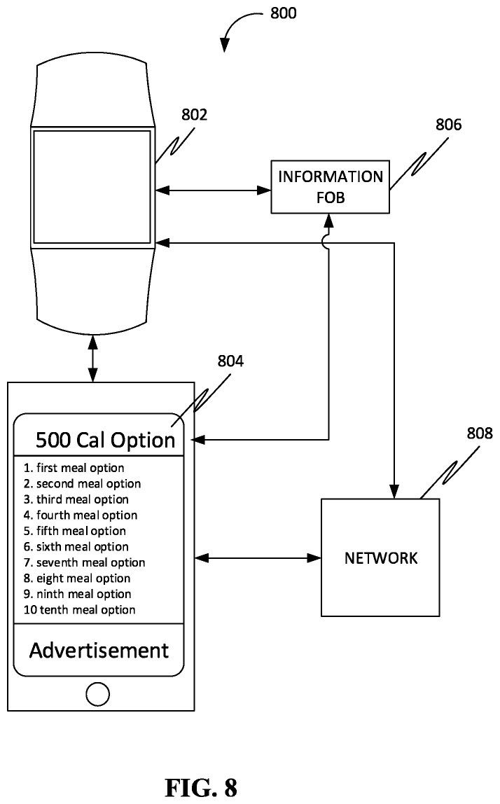

FIG. 8 is a block diagram of a system 800 for integrating technology for health and fitness, linking together health clubs, patrons, and restaurants for the benefit of all parties involved. The system 800 includes four key components: an interactive health watch 802, a smartphone application 804, an informational fob 806, and a network 808. The interactive health watch 802 is used by patrons of a health club, and may be used to offer several benefits inside the health club. While the smartphone application 804 offers some of the same benefits as the interactive health watch 802, the smartphone application 804 also provides some additional benefits. In addition, the smartphone application 804 is more accessible, since people frequently carry their phone at all times. The informational fob 806 is incorporated into weights, exercise machines, cycles etc. The informational fob 806 may be used with both personal and commercial equipment. Finally, the network 808 may provide a means for communications between the interactive health watch 802, the smartphone application 804, and the informational fob 806. In addition to the above, the use of the interactive health watch 802 and the smartphone application 804 may enable users to wirelessly check into a health club, forgoing the need for using the traditional member identification cards.

Further, the interactive health watch 802 is a personal apparatus that provides several benefits to a user. The interactive health watch 802 may include a heart rate monitor, an audio player (such as an MP3 player), a global positioning system (GPS), a nutrition tracker, near field communications capability (as well as Bluetooth and Wi-Fi), and network communications capability. In addition, the interactive health watch 802 may be able to count how many repetitions a user has performed, track an overall workout of a user, offer workout suggestions, and even display advertisements to the user. To provide these capabilities additional components may be needed within the interactive health watch 802. For example, a gyroscope and an accelerometer may be included and used to detect changes in position, allowing the interactive health watch 802 to perform the function of counting operations. The interactive health watch 802 may be active both during workouts and outside the health club. The heart rate monitor and repetition tracker may allow the interactive health watch 802 to receive and store data about a user's workout. For example, data about heart rate during a run or amount of repetitions performed during weightlifting may be stored and analyzed. In addition, user's entire workout may be stored, making the task of recording workout data effortless. As well as storing data locally, the interactive health watch 802 may communicate with the network 808 and related smartphone application 804, making data directly available through cloud storage. This information may then be used by the interactive health watch 802 to suggest future workouts. If a user is running on a treadmill, the heart rate monitor may provide data about heart rate, spanning across workouts. If the interactive health watch 802 detects that the heart rate is decreasing, a new workout may be suggested to increase heart rate. The recommendation may include increasing the speed of the treadmill, or something more complicated such as switching to a rowing machine. If so desired, a user may even be presented with multiple options, affording the user some flexibility in choosing the future workouts. The near-field communications and network communications may allow the interactive health watch 802 to receive additional workout data, providing the opportunity to recommend more specific routines to users.

While exercising is beneficial in the pursuit of good health, diet also plays a large role in personal health. To facilitate healthy eating, the nutrition tracker allows a user to easily record meals, whether by manual entry or importing data through near-field communications and network communications. This makes tracking total caloric and nutrient intake much easier, assisting users with achieving a target caloric intake and other goals, such as a certain split in calories from carbohydrates, protein, and fat. In addition to the above capabilities, the interactive health watch 802 includes an MP3 player, eliminating the need for a separate portable audio device. Many people prefer to exercise while listening to music. While the interactive health watch 802 may provide additional features, such as a timer function, care may be taken to balance capabilities with the likelihood of said capabilities being used by the majority of customers.

The smartphone application 804 provides many of the same capabilities as the interactive health watch 802 (such as a nutrition tracker) and communications ability with the network 808 and interactive health watch 802. In addition to the similarities with the interactive health watch 802, the smartphone application 804 provides several supplemental functions. These focus on assisting with nutrition, and include the ability to read barcodes (such as the quick response (QR) code) at restaurants, suggest potential healthy meals at restaurants, allow restaurants to advertise meal options that match a user's nutritional goals and provide directions to the restaurants. The participating restaurants may provide a menu that includes nutrition information for the individual dishes. This allows a user to access a list of meal choices and search for one that best matches their nutritional needs. Potentially, a user may also filter menu options by nutritional information such as total protein. The restaurants are also provided the opportunity to directly advertise to a user. The advertisements may be adjusted based on an individual user's current nutritional intake and target intake for the meal or day. For example, the nutritional tracking may only load advertisements for meals that match the targets for a user. This process may be automated, such that the restaurant does not need to directly access the user's data in order to provide relevant advertisements. In this manner, the user's privacy may be respected while still allowing for a mutually beneficial advertisement system. Once the user finds a suitable restaurant, whether by searching or through an advertisement, the smartphone application 804 may be used to provide directions to the restaurant. At the restaurant, the nutritional information from the meal may be automatically entered into the nutrition tracker when the user scans the corresponding barcode, which is placed on the menus and receipts of participating restaurants.

Further, the smartphone application 804 may be capable of scanning barcode information from participating consumer items. This applies mainly to groceries, which provide the option of scanning barcodes from receipts to automatically enter nutritional data into a user's daily consumption. As well as providing barcodes on receipts, pre-packaged goods may have barcodes built into the packaging. The prime candidates for these barcodes include frozen meals, prepared sandwiches, canned goods, and even energy bars. This allows a user to simply scan their frozen dinner or post-workout protein bar and have the information automatically uploaded to their nutritional profile.

The informational fobs (such as the informational fob 806) may be installed throughout the health club, being integrated into machines, free weights, and even specific rooms. The informational fob 806 may be capable of communicating with both the interactive health watch 802 and the smartphone application 804. The informational fob 806 may store the information specific to its location, and transmit that information to the interactive health watch 802 or the smartphone application 804 as required. The informational fob 806 may include a radio-frequency identification (RFID) tag, which may be used in communication with the interactive health watch 802 and the smartphone application 804. The RFID tag helps users, especially those who are just beginning to exercise, to select different exercises and classes, as well as store relevant workout information via the interactive health watch 802, the smartphone application 804, and the cloud storage. For example, a user might use their interactive health watch 802 or the smartphone application 804 to identify a leg press machine. The RFID tag may report back with basic information, such as what the machine is and potentially some basic pointers on how to use it. The user may then be asked if they want to use the leg press; if they choose to do so then information from the RFID tag may be entered in the user's workout log, including the number of repetitions performed. Likewise, the informational fob 806 may allow the user to easily record when they take part in a group class, such as yoga or cycling, and have the relevant information automatically entered into their workout log.

Further, the network 808 may provide integration across the entire health club. The network 808 may provide several benefits, to both patrons and ownership. The network 808 may track incoming and outgoing traffic from users, at which point it may automatically upload relevant information from patrons' interactive health watch 802 and smartphone. This is beneficial if an outside workout is done, such as an outdoors run. The next time a patron enters the health club, their information may be updated in the network 808, including recording their outside run in their workout history. Alternatively, the details about the outside workout may be shared with the network 808 via the patron's health watch 802 and/or smartphone via the Internet.

Similarly, upon exiting the health club, the network 808 may receive data regarding the patron's usage of the health club during that visit, including exercises performed, classes attended, and machines used. In addition to the automated entry, the network 808 may also be accessed from the Internet, allowing the patrons to manually enter and update exercise information. Further, the network 808 (as well as the interactive health watch 802 and the smartphone application 804) may communicate with electronic workout devices. For example, a large variety of workout games may be available through current entertainment consoles, including dance games and exercise games utilizing motion detection. These devices may be capable of communicating with the network 808 over the Internet, while they may communication with the interactive health watch 802 and the smartphone application 804 through shorter range methods, such as Bluetooth, Wi-Fi, and even USB connections.

The network 808 may provide an additional benefit for the health club. Since member usage is easily monitored by means of the interactive health watch 802, the smartphone application 804, and informational fob 806, the health club may effortlessly perform an internal review. The health club may see which machines and classes are popular and heavily used, as well as machines/classes that have low usage. For example, data might show that cycling classes are rapidly filled up, whereas boxing classes rarely fill up, or take a long time to do so. As a result, the health club may offer less boxing classes in favor of increasing the number of cycling classes available. As another example, the data may show that the leg press machine is being heavily utilized; this may provide a justification to acquire additional leg press machines, or even indicate that the leg press machine needs to undergo maintenance.

As described above, the existing health clubs may be upgraded by retrofitting the disclosed system. Further, additional options may be provided for home gyms and new commercial installations. For home gyms, personal workout equipment (e.g. kettlebells, pull-up bars, and other small apparatuses) may be built with included RFID tags. For commercial installations, the computing systems may be installed directly into exercise equipment, effectively integrating the capabilities of the informational fob 806 into said equipment. For preexisting health clubs, the informational fob 806 may need to be provided for existing machinery, which may gradually be phased out as the equipment reaches end-of-life.

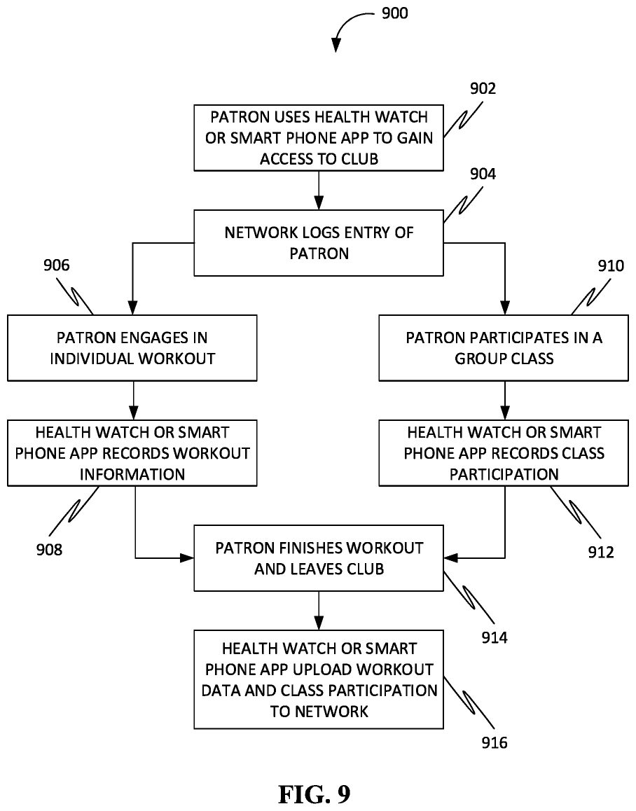

FIG. 9 is a flowchart of a method 900 for recording a workout in a gym in accordance with an exemplary embodiment. At 902, a patron may use health watch or smartphone application to gain access to a health club. At 904, a network may log entry of patron. At 906, the patron may engage in an individual workout. At 908, the health watch or smartphone application may record workout information. At 910, the patron may participate in a group class. At 912, the health watch or smartphone application may record the class participation. At 914, the patron may finish the workout and leave the club. Thereafter, at 916, the health watch or smartphone application may upload workout data and class participation to the network.

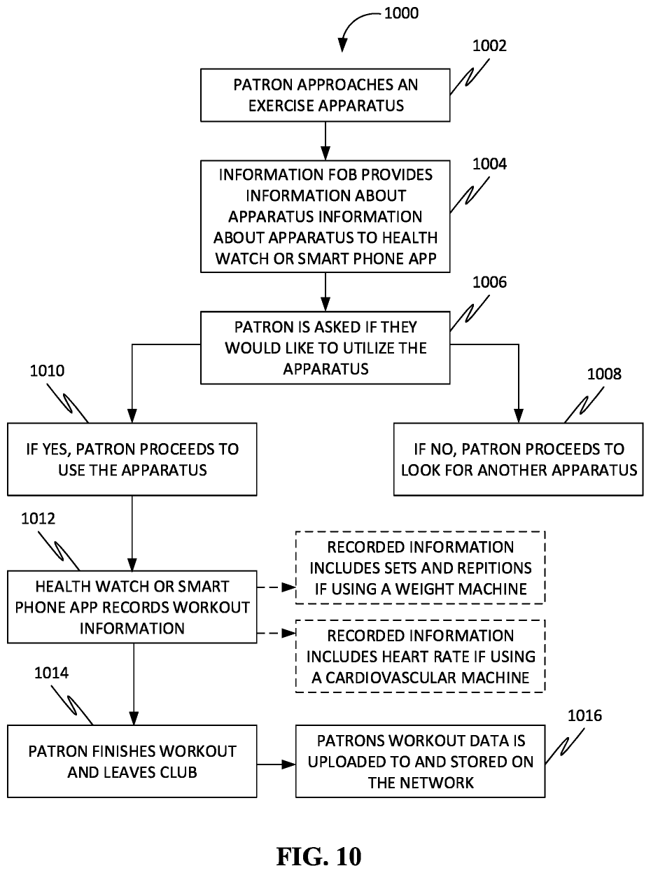

FIG. 10 is a flowchart of a method 1000 for using gym equipment in accordance with an exemplary embodiment. At 1002, the patron may approach an exercise apparatus. At 1004, an information fob may provide information about apparatus to a health watch or smartphone application. Then at 1006, the patron may be asked if they would like to utilize the apparatus. If the patron does not want to use the apparatus, then the patron may proceed to look for another apparatus at 1008. However, if the patron wants to use the apparatus, then the patron may proceed to use the apparatus at 1010. At 1012, the health watch or smartphone application may record workout information. The recorded information may include sets and repetitions if using a weight machine. Further, the recorded information may include heart rate if using a cardiovascular machine. Then, at 1014, the patron may finish the workout and leave the club. Thereafter, at 1016, the patron's workout data is uploaded to and stored on the network.

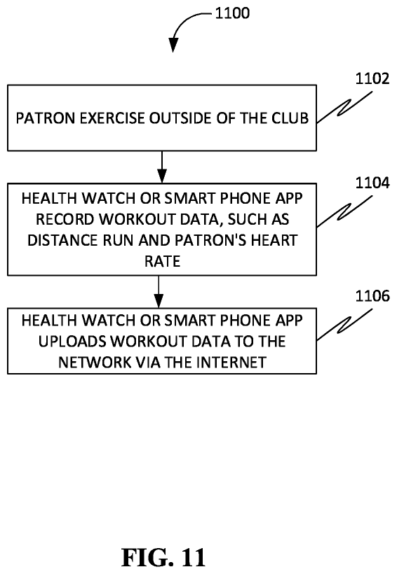

FIG. 11 is a flowchart of a method 1100 for recording workout outside a gym in accordance with an exemplary embodiment. At 1102, the patron exercise outside of the club. At 1104, the health watch or smartphone application records workout data, such as distance run and patron's heart rate. At 1106, the health watch or smartphone application uploads workout data to the network/cloud via the patron's health watch and/or smartphone via the Internet. Alternatively, the health watch or smartphone application may upload the workout data when the patron enters the gym.

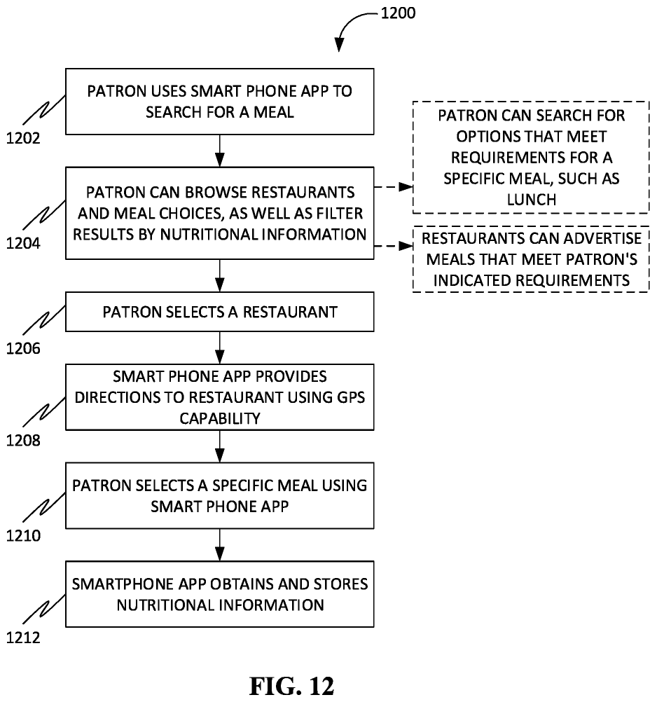

FIG. 12 is a flowchart of a method 1200 for providing food choices to a user in accordance with an exemplary embodiment. At 1202, the patron may use the smartphone application to search for a meal. At 1204, the patron may browse restaurants and meal choices, as well as filter results by nutritional information. The patron may search for options that meet requirements for a specific meal, such as lunch. Further, the restaurants may advertise meals that meet patron's indicated requirements. At 1206, the patron may select a restaurant. At 1208, the smartphone application may provide directions to the restaurant using GPS. At 1210, the patron may select a specific meal from the smartphone application. Further, the patron may order the selected meal using the smartphone application before arriving at the restaurant. Then, at 1212, the smartphone application may obtain and store the nutritional information based on the selected meal. For example, the smartphone application may obtain the nutritional information from a nutritional database. Further, the data may be uploaded to the network via the Internet.

Alternatively, at the restaurant, the patron may scan barcode from menu or receipt using smartphone application. Further, the smartphone application may read and store nutritional information from the barcode. Then, the data may be uploaded to the network next time patron enters the health club.

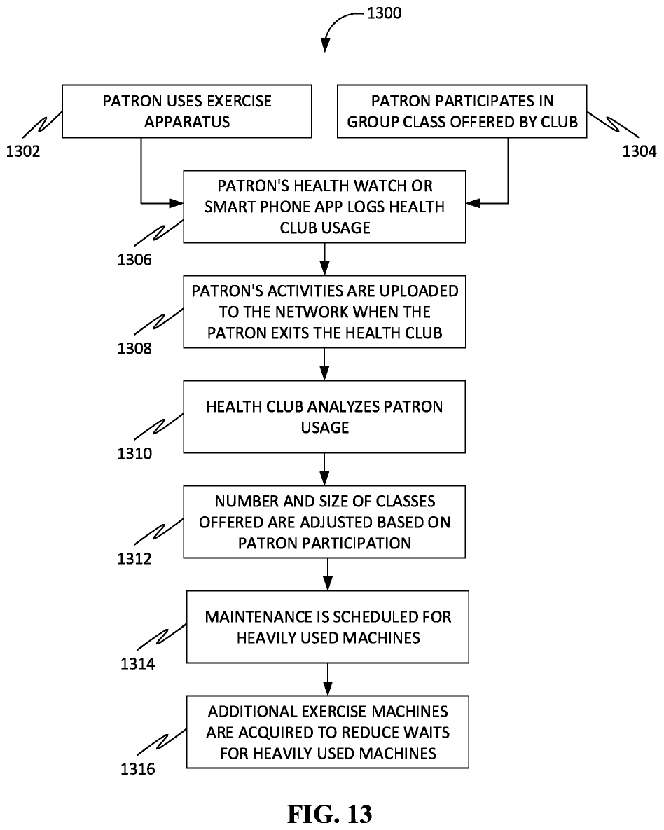

FIG. 13 is a flowchart of a method 1300 for customizing workouts for a user in accordance with an exemplary embodiment. At 1302, the patron may use exercise apparatus. At 1304, the patron may participate in group class offered by the club. Accordingly, at 1306, the patron's health watch or smartphone application may log health club usage. At 1308, the patron's activities may be uploaded to the network when the patron exits the health club. At 1310, the health club may analyze patron usage. At 1312, the number and size of classes offered may be adjusted based on patron participation. Thereafter, at 1314, maintenance is scheduled for heavily used machines. Further, additional exercise machines may be acquired to reduce waits for heavily used machines at 1316.

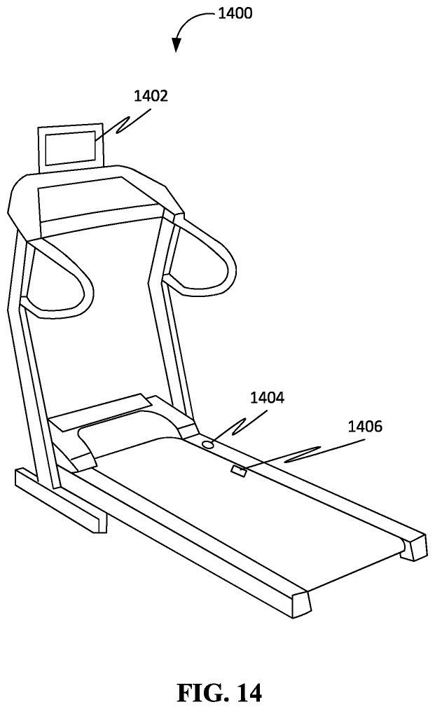

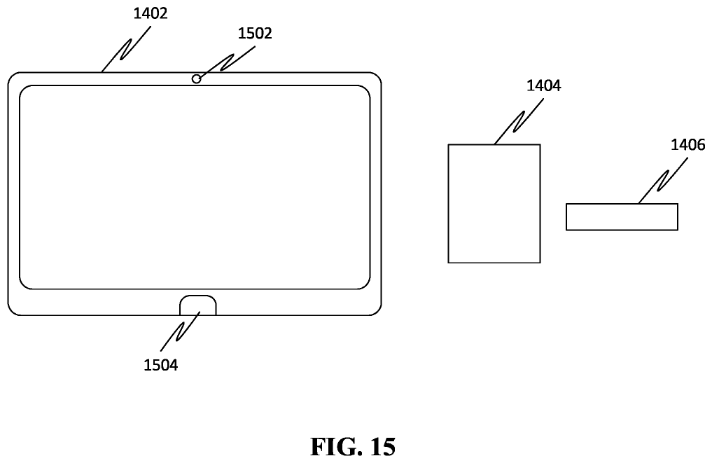

FIG. 14 illustrates a treadmill 1400 that may be placed in a health club in accordance with an exemplary embodiment. The treadmill 1400 may include a screen 1402, a fob 1404 and a sensor 1406. FIG. 15 shows the screen 1402, the fob 1404 and the sensor 1406 in further detail. The screen 1402 may include a camera 1502 and a fingerprint scanner 1504. The screen 1402 may further include one or more of a camera, an active ultra-bandwidth RFID, a fingerprint scanner, a gyroscope, an accelerometer, facial recognition capability, Wi-Fi capability, Bluetooth capability, NFC capability. The fob 1404 may further include Wi-Fi capability, Bluetooth capability, NFC capability, an active ultra-bandwidth RFID, a gyroscope, and an accelerometer.

The sensor 1406 may be of the thickness of a sticker. The sensor 1406 may be attached to a belt of the treadmill 1400 to be able to determine speed and distance to automatically load into the exercise journal and calculate calories burned.

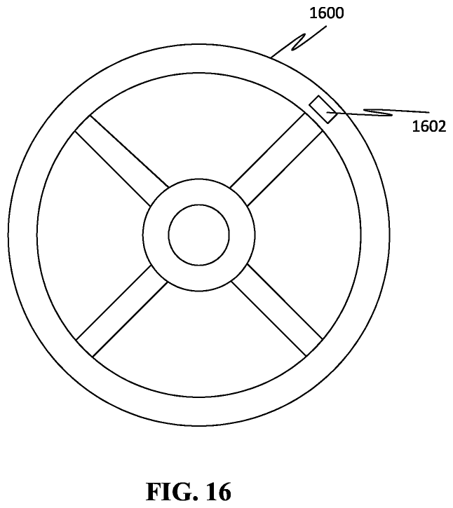

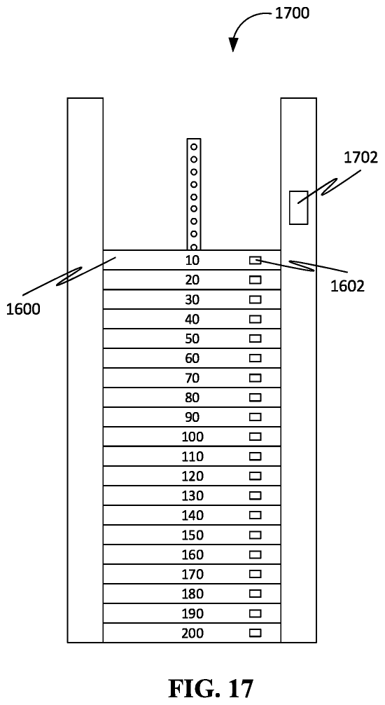

The fobs and sensors are installed on equipment as shown in FIGS. 16-18 below. FIG. 16 illustrates a weight plate 1600 tagged with a sensor 1602 in accordance with an exemplary embodiment. FIG. 17 illustrates plates loaded machine 1700 with a fob 1702 in accordance with an exemplary embodiment. The fob 1702 will be able to identify how much is being loaded by communication with one or more sensors (such as the sensor 1602 on the plate 1600). Further, the fob 1702 determines how much weight is being used and the repetitions and sets as the sensor 1602 passes by the fob 1702.

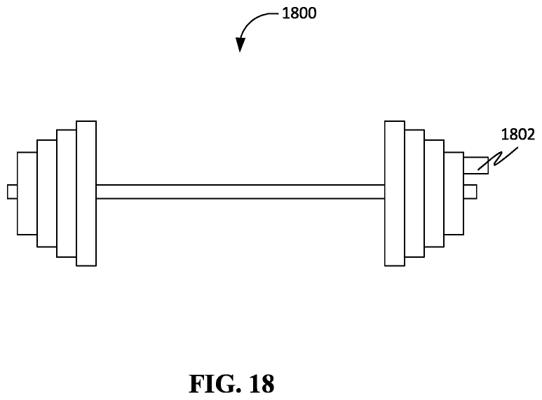

FIG. 18 illustrates a dumbbell 1800 with a fob 1802 in accordance with an exemplary embodiment. The dumbbells (with preloaded weights) may only have a fob. Since the weight is already determined the fob 1802 may simply count repetitions and sets.

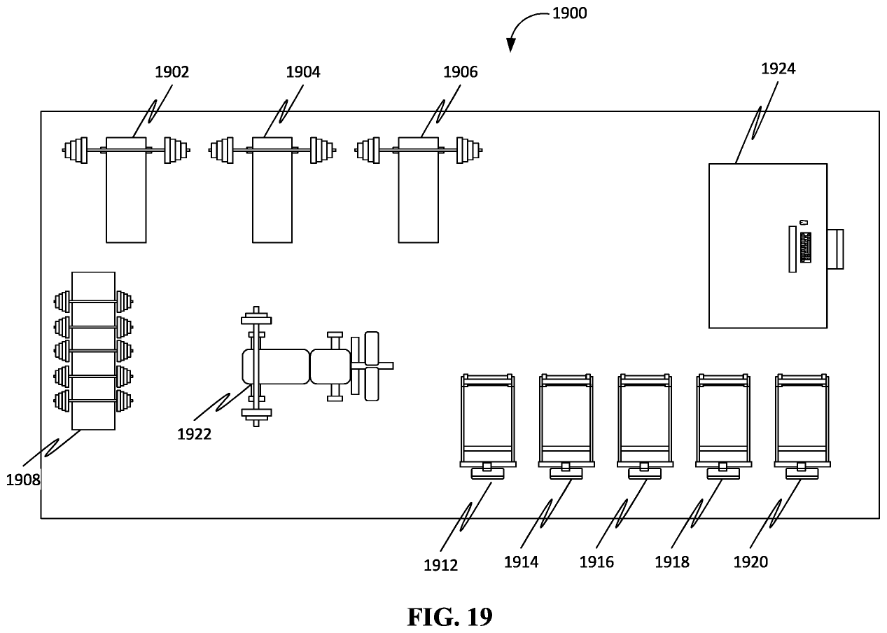

FIG. 19 illustrates a floor map 1900 of a gym in accordance with an exemplary embodiment. The system 200 may be used to identify where equipment is located and to direct the user to the equipment. Further, the system 200 may also indicate if the equipment is in use and what else is available. The equipment may include dumbbell bench presses 1902-1906, a weight rack 1908, treadmills 1912-1920, and chest press 1922 placed at a specific location in the gym. Further, the gym may include a server room 1924.

Each piece of equipment and weight will be identified by the system 200 allowing the gym owners and staff to identify if and which members are not re-racking or putting away weights/equipment. Then the staff may warn or speak with the identified members. This is a massive problem and frustration for gym goers and owners when people do not re-rack or put things away properly.

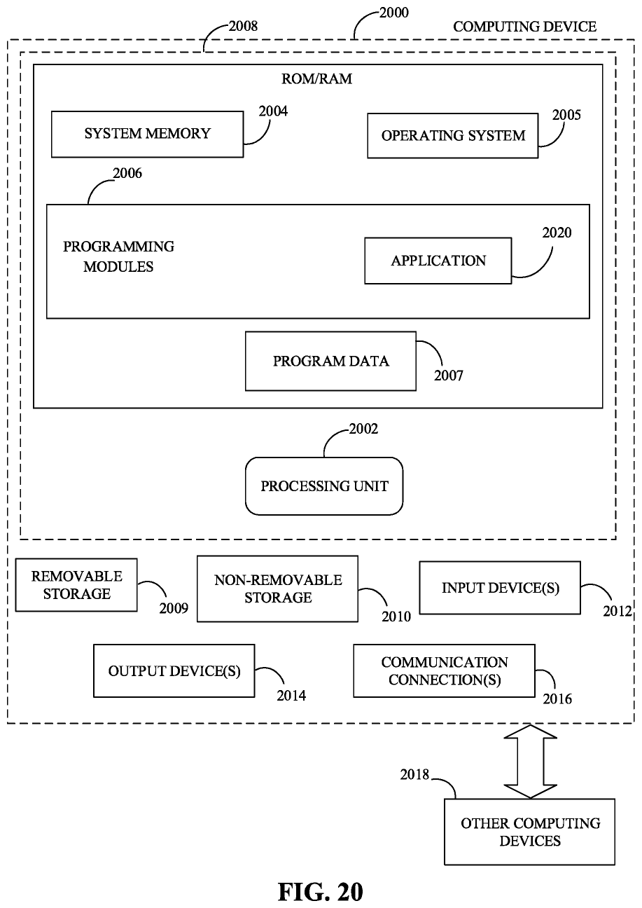

With reference to FIG. 20, a system consistent with an embodiment of the disclosure may include a computing device or cloud service, such as computing device 2000. In a basic configuration, computing device 2000 may include at least one processing unit 2002 and a system memory 2004. Depending on the configuration and type of computing device, system memory 2004 may comprise, but is not limited to, volatile (e.g. random-access memory (RAM)), non-volatile (e.g. read-only memory (ROM)), flash memory, or any combination. System memory 2004 may include operating system 2005, one or more programming modules 2006, and may include a program data 2007. Operating system 2005, for example, may be suitable for controlling computing device 2000's operation. In one embodiment, programming modules 2006 may include a machine learning module. Furthermore, embodiments of the disclosure may be practiced in conjunction with a graphics library, other operating systems, or any other application program and is not limited to any particular application or system. This basic configuration is illustrated in FIG. 20 by those components within a dashed line 2008.

Computing device 2000 may have additional features or functionality. For example, computing device 2000 may also include additional data storage devices (removable and/or non-removable) such as, for example, magnetic disks, optical disks, or tape. Such additional storage is illustrated in FIG. 20 by a removable storage 2009 and a non-removable storage 2010. Computer storage media may include volatile and nonvolatile, removable and non-removable media implemented in any method or technology for storage of information, such as computer-readable instructions, data structures, program modules, or other data. System memory 2004, removable storage 2009, and non-removable storage 2010 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 2000. Any such computer storage media may be part of device 2000. Computing device 2000 may also have input device(s) 2012 such as a keyboard, a mouse, a pen, a sound input device, a touch input device, a location sensor, a camera, a biometric sensor, etc. Output device(s) 2014 such as a display, speakers, a printer, etc. may also be included. The aforementioned devices are examples and others may be used.

Computing device 2000 may also contain a communication connection 2016 that may allow device 2000 to communicate with other computing devices 2018, such as over a network in a distributed computing environment, for example, an intranet or the Internet. Communication connection 2016 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 2004, including operating system 2005. While executing on processing unit 2002, programming modules 2006 (e.g., application 2020 such as a media player) may perform processes including, for example, one or more stages of methods, algorithms, systems, applications, servers, databases as described above. The aforementioned process is an example, and processing unit 2002 may perform other processes. Other programming modules that may be used in accordance with embodiments of the present disclosure may include machine learning application etc.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general purpose graphics processor-based systems, multiprocessor systems, microprocessor-based or programmable consumer electronics, application specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

Although the invention has been explained in relation to its preferred embodiment, it is to be understood that many other possible modifications and variations can be made without departing from the spirit and scope of the invention.

What is claimed is:

1. A system for facilitating monitoring of fitness devices, the system comprising:
a plurality of sensors configured to be attached to a plurality of fitness devices, wherein the plurality of sensors is configured for generating sensor data representative of at least one state of the plurality of fitness devices;
a plurality of transceivers communicatively coupled with the plurality of sensors, wherein the plurality of transceivers is further configured for communicating with each of a portable electronic device and a server computer;
a plurality of local processing devices communicatively coupled to each of the plurality of sensors and the plurality of transceivers, wherein the plurality of local processing devices is further configured for processing the sensor data; and
a server computer comprising:
a server communication device configured for communicating with the at least one transceiver, wherein the communicating comprises receiving the sensor data;
a server processing device communicatively coupled with the server communication device, wherein the server processing device is configured for:
analyzing the sensor data;
generating at least one insight corresponding to at least one fitness device of the plurality of fitness devices based on the analyzing of the sensor data; and
a server storage device communicatively coupled to the server processing device, wherein the server storage device is configured for storing the sensor data and the at least one insight in association with a plurality of fitness device identifiers associated with the plurality of fitness devices.

2. The system of claim 1 further comprising a plurality of display devices communicatively coupled to the plurality of processing devices, wherein the plurality of display devices is configured for displaying fitness data associated with the portable electronic device, wherein the plurality of transceivers is configured for receiving the fitness data from the portable electronic device.

3. The system of claim 2, wherein a local processing device of the plurality of local processing devices is configured for:
determining a usage time of a fitness device corresponding to the local processing device; and
generating a user waiting message based on the usage time exceeding a predetermined time period and receipt of a usage request, wherein a transceiver of the plurality of transceivers corresponding to the fitness device is further configured for receiving the usage request from at least one of the server computer and at least one other transceiver of the plurality of transceivers, wherein a display device of the plurality of display devices corresponding to the fitness device is further configured for displaying the user waiting message.

4. The system of claim 1, wherein the at least one state of the plurality of fitness devices comprises at least one of an operational state and a usage state.

5. The system of claim 1 further comprising a plurality of physiological sensors corresponding to the plurality of fitness devices communicatively coupled to the plurality of processing devices, wherein the plurality of physiological sensors is configured for generating physiological data corresponding to at least one user, wherein a physiological sensor corresponding to a fitness device is configured for generating physiological data of a user of the fitness device during usage of the fitness device, wherein the plurality of transceivers is configured for transmitting the physiological data to at least one of the portable electronic device and the server computer.

6. The system of claim 5, wherein the plurality of transceivers is configured for transmitting the physiological data to the server computer, wherein the server processing device is further configured for analyzing the physiological data, wherein generating the at least one insight is further based on the analyzing of the physiological data, wherein the server storage device is further configured for storing the physiological data in association with a user identifier associated with the user and a fitness device identifier associated with the fitness device.

7. The system of claim 1 further comprising a plurality of biometric sensors corresponding to the plurality of fitness devices communicatively coupled to the plurality of local processing devices, wherein the plurality of biometric sensors is configured for generating biometric data associated with at least one user of the plurality of fitness devices, wherein the plurality of transceivers is configured for transmitting the biometric data to the server computer.

8. The system of claim 7, wherein the plurality of transceivers is further configured for:
receiving user identity data from the portable electronic device;
transmitting the user identity data to the server computer;
transmitting the biometric data associated with a user to the server computer; and
receiving an authentication result from the server computer, wherein the server processing device is configured for authenticating the user based on the biometric data and the user identity, wherein the generating of the at least one insight is further based on the authenticating.

9. The system of claim 1, wherein the plurality of transceivers is configured for transmitting a check-in indicator to the server computer based on the communicating between the portable electronic device and at least one transceiver of the plurality of transceivers, wherein the check-in indicator represents entry of a user associated with the portable electronic device into a facility housing the plurality of fitness devices.

10. The system of claim 1, wherein the portable device comprises a wearable computing device, wherein the wearable computing device comprises at least one activity sensor configured for generating at least one physical activity data corresponding to a user wearing the wearable computing device, wherein the plurality of transceivers is further configured for receiving the at least one physical activity data and transmitting the at least one physical activity data to the server computer, wherein the generating of the at least one insight is further based on the at least one physical activity data.

11. The system of claim 10, wherein the wearable computing device further configured for storing the at least one physical data corresponding to a first time period, wherein the sensor data corresponds to a second time period, wherein the first time period and the second time period are non-identical, wherein the generating of the at least one insight is further based on each of the first time period and the second time period.

12. The system of claim 1, wherein the plurality of transceivers is further configured for:
receiving a workout recommendation from the server computer, wherein the server processing device is further configured for generating the workout recommendation based on the at least one insight; and
transmitting the workout recommendation to the portable electronic device, wherein the portable electronic device is further configured for presenting the workout recommendation.

13. The system of claim 1 further comprising a plurality of local storage devices corresponding to the plurality of fitness devices, wherein the plurality of local storage devices is communicatively coupled to the plurality of local processing devices, wherein each local storage device of the plurality of local storage devices is configured for storing at least one of a location of a corresponding fitness device within a facility housing the plurality of fitness devices and a fitness device identifier of the corresponding fitness device.

14. The system of claim 13 further comprising a plurality of location sensors corresponding to the plurality of fitness devices, wherein the plurality of location sensors is communicatively coupled to the plurality of local processing devices, wherein each location sensor of the plurality of location sensors is configured for generating location of a corresponding fitness device.

15. The system of claim 13, wherein the plurality of local storage devices is further configured for storing a plurality of fitness device usage data, wherein the plurality of transceivers is further configured for transmitting the plurality of fitness device usage data to the portable device, wherein a fitness device usage data corresponding to a fitness device comprises information pertaining to usage of the fitness device.

16. The system of claim 13, wherein the plurality of transceivers is configured for receiving a fitness goal from the portable electronic device, wherein the plurality of local processing devices is configured for generating a fitness device guidance data based on the fitness goal, wherein the plurality of transceivers is further configured for transmitting the fitness device guidance data to the portable electronic device, wherein a fitness device guidance data corresponding to a fitness device comprises a location of the fitness device within the facility housing the plurality of fitness devices.

17. The system of claim 1, wherein the at least one insight comprises at least one facility recommendation.

18. The system of claim 17, wherein the at least one facility recommendation comprises a maintenance recommendation corresponding to at least one fitness device of the plurality of fitness devices.

19. The system of claim 18, wherein the server processing device is further configured for generating a maintenance request based on the maintenance recommendation, wherein the server communication device is further configured for transmitting the maintenance request to a service technician device.

20. The system of claim 1 further comprising a plurality of facility display devices installed within the facility housing the plurality of fitness devices, wherein the plurality of facility display devices is configured for receiving display content from the server computer, wherein the server processing device is further configured for generating the display content based on the at least one insight, wherein the at least one insight comprises a traffic of a plurality of users within a facility housing the plurality of fitness devices.
